(19) United States
(12) Patent Application Publication
(10) Pub. No.: US 2025/0250522 A1
(43) Pub. Date: Aug. 7, 2025

(54) Recapitulating Tissue-Native Architectures in Bio-printable Hydrogels

(71) Applicant: University of Cincinnati, Cincinnati, OH (US)

(72) Inventors: Riccardo Barrile, Crescent Springs, KY (US); Li Cai, Hebron, KY (US)

(73) Assignee: University of Cincinnati, Cincinnati, OH (US)

(21) Appl. No.: 18/856,845

(22) PCT Filed: Apr. 14, 2023
(86) PCT No.: PCT/US2023/018662

§ 371 Date: Oct. 14, 2024

Related U.S. Application Data
(60) Provisional application No. 63/331,213, filed on Apr. 14, 2022.

(51) Int. Cl. C12M 3/00; B33Y 10/00; B33Y 80/00; C12M 1/12; C12M 3/06; C12N 5/071; C12N 5/0793; C12N 5/09; G03F 7/26

(52) U.S. Cl. CPC C12M 21/08; B33Y 10/00; B33Y 80/00; C12M 23/16; C12M 25/14; C12N 5/0619; C12N 5/069; C12N 5/0693; G03F 7/26; C12N 2533/54

(57) ABSTRACT

A device for modelling physiological and pathophysiological states of the brain is provided. The device comprises a hydrogel with micropattern channels having a length, width and depth. The micropattern channels comprise cells selected from the group consisting of neurons, astrocytes, microglia, oligodendrocytes, neuronal organoids, cancer cells, cancer spheroids, brain tumoral cells and combinations thereof.

### CROSS-REFERENCE TO RELATED APPLICATIONS

This application is a continuation of PCT Application No. PCT/US23/18662 filed Apr. 14, 2023, which claims the benefit of the filing date of, U.S. Patent Application Ser. No. 63/331,213, filed on Apr. 14, 2022, the disclosures of which are incorporated by reference herein in their entirety.

### TECHNICAL FIELD

The present invention relates to in vitro tissue and organ models.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The 2013 Global Burden of Disease (GBD) Study found that the number of infectious and nutritional disorders had decreased between 1990 and 2013, but noncommunicable diseases such as stroke, heart disease, cancer, and diabetes had increased. Nearly 800,000 people have a stroke in the United States each year with approximately 140,000 of the stroke patients dying. The GBD Study also found that although stroke incidence, prevalence, mortality, and disability-adjusted life-years seemed to decline between 1990 and 2013, the overall stroke burden in terms of absolute numbers of people affected by or experience long-term disability from stroke increased globally in both sexes and in all ages. The long-term disability costs of stroke survivors have an estimated value of $33.9 billion, so the prevention and treatment of strokes is of great interest for clinicians.

There are two main types of strokes: ischemic and hemorrhagic. Ischemic strokes occur when blood flow to the brain is reduced or stopped, often due to a clot, which causes permanent cell damage due to the deprivation of oxygen during the stoke. Hemorrhagic strokes occur when a blood vessel ruptures and blood leaks into the brain. Both types of strokes disrupt the blood brain barrier (BBB) and can have long-term effects on survivors such as paralysis, headaches, impaired coordination, short-term memory, and vision trouble. Ischemic strokes account for 87% of all strokes.

The BBB refers to the unique properties of the central nervous system (CNS) which includes the tight regulation of transportation of molecules into and out of the CNS. Strokes are known to cause the structural disruption of the endothelial cells' (ECs) tight junctions due to cell damage caused by the deprivation of oxygen of cells during the ischemic stroke. The disruption of the BBB's permeability is what leads to many of the long-term effects after strokes. The disturbance of the tight junctions results in increased permeability which allows fluid to enter the brain and cause edema. The increase in permeability of the BBB also results in inflammation within the brain which can further reduce the integrity of the BBB. This disruption of the BBB may persist after the stroke and cause long term complications including an increased risk of a second stroke.

Current treatments for strokes include drugs and surgical procedures. For ischemic strokes, an injection of tissue-type plasminogen activator (tPA) is the gold standard treatment. tPA breaks up the clot that causes the stroke, restoring oxygen to the brain, and is the only Food and Drug Agency (FDA) approved treatment for ischemic strokes. tPA can break up clots if administered within three hours after stroke onset without requiring an invasive procedure; the quick removal of the clot can allow patients to recover from the stroke faster. For larger clots that tPA cannot dissolve completely, a stent may be used to directly remove the clot. However, tPA's side effects include the opening of the BBB and neurotoxicity which contribute to possible long-term complications for patients.

In order to better understand what happens to the BBB during stroke, many groups have been developing models of the BBB including murine models and various in vitro models. However, current in vivo and in vitro models have their downfalls. Murine models have been used to study BBB transport mechanisms, but experiments can be expensive and some drugs that are designed to work in mice models fail in humans. In vitro models can be limited if they are unable to properly mimic the natural three dimensional (3D), highly organized, dynamic BBB. Therefore, a need still exists for improved methods to understand the blood brain barrier.

Microphysiological systems and Organ-on-Chips are emerging as attractive alternatives to animal models and various microfabrication techniques often based on soft-lithographic methos have been developed to prototype a growing number of models. Bioprinting is one technique that has been used in recent years as a potential alternative to traditional soft-lithography as it provides a mean to generate biocompatible scaffolds where cells can function and in a highly scalable manners. Current bioprinting methods, however, are limited in their ability to recapitulate functional architecture of human brain.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, a device for modelling physiological and pathophysiological states of the brain is provided. The device comprises a hydrogel with micropattern channels having a length, width and depth. The micropattern channels comprise cells selected from the group consisting of neurons, astrocytes, microglia, oligodendrocytes, neuronal organoids, cancer cells, cancer spheroids, brain tumoral cells and combinations thereof.

In one embodiment, the cells are either of primary origin or derived from stem cells. In another embodiment, the micropattern channels comprise human neurons and the human neurons form a synaptic network. In one embodiment, the human neurons form a synaptic network that is co-cultured with tumoral cells, wherein the tumoral cells are human or mice tumoral cells. In another embodiment, the micropattern channels further comprise endothelial cells. In one embodiment, one or more of the micropattern channels have inlets that allow for direct access to the channels with conventional micropipettes. In another embodiment, the hydrogel comprises parallel microfluidic chambers. In one embodiment, the hydrogel comprises multiple parallel microfluidic chambers.

In another aspect of the present invention, a device for modelling physiological and pathophysiological states of the brain such as blood-brain barrier function and neuronal activity is provided. The device comprises human neurons or brain cancer cells (glioblastoma) seeded on a micropatterned hydrogel. In one embodiment, the human neurons form a synaptic network. In another embodiment, the cancer cells adhere and spread onto the same surface and therefore the system allows for co-culturing of healthy and diseased human cells what represents the natural organization of brain cells in a tumoral tissue where usually healthy cells grow next to cancer. In another embodiment, the hydrogel is selected from the group consisting of Gelatin and hyaluronic acid (Gelatin-HAMA), Gelatin-Cellulose, HAMA-Gelatin-Cellulose, HAMA-Cellulose-Gelatin-trans glutaminase (TG), GelMA-Cellulose-Gelatin-TG, and GelMA-HAMA-Gelatin-TG. In one embodiment, the hydrogel is GelMA-HAMA-Gelatin-TG.

In another embodiment, the hydrogel material further comprises a material that improves cell adhesion. In one embodiment, the hydrogel material further comprises Matrigel, Collagen, Fibronectin or other cell-adhesive molecules. In another embodiment, the hydrogel comprises micropattern channels having a length, width and depth. In one embodiment, the micropattern channels have a width of less than about 20 μm. In another embodiment, the micropattern channels have a width of less than about 10 μm.

In another aspect of the present invention, a method of making a micropatterned hydrogel is provided. The method involves printing a hydrogel material on a substrate; then positioning a photomask over the hydrogel material; exposing the photomask and hydrogel to UV light; and removing the photomask. Micropattern channels are created on the surface of the hydrogel material. The micropattern channels having a length, width and depth.

In one embodiment, the hydrogel is selected from the group consisting of Gelatin-HAMA, Gelatin-Cellulose, HAMA-Gelatin-Cellulose, HAMA-Cellulose-Gelatin-TG, GelMA-Cellulose-Gelatin-TG, and GelMA-HAMA-Gelatin-TG. In another embodiment, the hydrogel material further comprises a material that improves cell adhesion. In one embodiment, the hydrogel material further comprises Matrigel, collagen, fibronectin, other biomolecules or synthetic peptides or combinations thereof. In one embodiment, the hydrogel material further comprises Matrigel. In another embodiment, the hydrogel material is bioprinted.

In another aspect of the present invention, a method of making a neuronal activity model is provided. The method involves making a micropatterned hydrogel as described above; and seeding human neurons in one or more of the micropattern channels. In one embodiment, the human neurons form a synaptic network.

In another aspect of the present invention, a method of making a micropatterned hydrogel is provided. The method involves printing a hydrogel material on a substrate; and applying a sacrificial bioink to the hydrogel material. The sacrificial bioink creates micropattern channels on the surface of the hydrogel material. The micropattern channels having a length, width and depth. In one embodiment, the sacrificial bioink is Pluronic or gelatin. In another embodiment, the hydrogel is GelMA-HAMA-Gelatin-TG.

In another aspect of the present invention, the apical surface can be patterned using UV-light or a simple stamp to obtain complex geometry and to guide tissue structure and function. A microchannel can be generated using sacrificial material. In another embodiment endothelial cells can be cultured in the micropatterned channel while neurons and cancer cells or neurons and astrocytes or other cells can be cultured on the apical surface of the scaffold. In one embodiment, a device for modelling neuronal activity is provided. The device comprises a hydrogel with micropattern channels having a length, width and depth. The micropattern channels comprise endothelial cells. Also, the hydrogel has an apical surface and the apical surface comprises cells selected from the group consisting of neurons, astrocytes, microglia, oligodendrocytes, neuronal organoids, cancer cells, cancer spheroids, brain tumoral cells and combinations thereof. In another embodiment, the endothelial cells form a tight monolayer and physiologically relevant barrier function and can be perfused with cell culture medium or blood.

In another aspect of the present invention, multiple parallel microfluidic channels can be generated using sacrificial material or UV light or lasers. In one embodiment the channels are generated inside the hydrogel. In another embodiment channels can have inlets that allow for direct access to the channels with conventional micropipettes. In one embodiment one or more channels can be seeded with endothelial cells and perfused with cell culture medium or blood to mimic the blood vessels that perfuse the brain. One or more channels can be seeded with brain cells such as neurons, astrocytes, microglia, oligodendrocytes that can be of primary origin or derived from stem cells to reconstitute the neuronal compartment of the human brain. In another embodiment cancer cells or cancer spheroids or brain tumors obtained from a patient resections can be encapsulated or pipetted in the channel to generate a brain tumor model.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1 is a schematic of the basic process of the present invention for preparing hydrogels for printing and patterning.

FIG. 2A is the chemical structure of crosslinked GelMA hydrogel.

FIG. 2B is the chemical structure of hyaluronic acid methacrylate (HAMA).

FIG. 2C is the chemical structure of cellulose.

FIG. 2D is the chemical structure of gelatin.

FIG. 2E is the chemical structure of fibronectin.

FIG. 3 is a schematic of a micropatterning method according to the present invention.

FIG. 4 is an image of a micropatterned HAMA-GelMA-Matrigel hydrogel.

FIG. 5 is an image showing the four dimensions measured for a node-crosshair structure. All four crosshairs' lengths and widths are measured for each node-crosshair structure.

FIG. 6 is a schematic of a photomask pattern with dimensions.

FIG. 7 is a schematic of a hydrogel and seeding setup for neurons and astrocytes according to the present invention.

FIG. 8 is a graph showing the printability of various hydrogel formulations.

FIG. 9 is a graph showing cell tracker red staining of U87 MG cells on hydrogels looking at viability (day 4).

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein “apical” means the top of a shape or object.

As used herein, “micropattern” means creating precise and controlled microscale patterns on surfaces to direct the behavior and organization of cells. This technique can be used to create complex tissue structures that mimic natural tissue organization and function.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention involves the discovery that human neurons seeded on micropatterned hydrogels are viable and display increased and controlled synaptic network formation and functionality compared to cells seeded on traditional non-patterned hydrogels. The present invention provides a method to recreate patterns that have been shown to guide synapse formation with hydrogels and to demonstrate that the micropatterned hydrogel promotes the formation of an organized network of functional synapses compared to non-patterned hydrogels. The method is useful for creating patterns in a BBB OOC model to study strokes. The directionality of neuronal signaling during strokes is not well understood. Applying the photopatterning technique to the BBB OOC allows for distinct synaptic regions to be formed which will allow the neuronal signaling to be seen in real time in the model. While this technique can be universally applied to neuronal models to guide synapse formation, the application of guiding synapses in the stroke BBB OOC model is meant to provide insight into the signaling that occurs in normal vs post-stroke tissue.

The novel UV-patterning approach of the present invention enables the generation of 3D-microstructures with a level of precision and resolution (smaller than 20 μm) that traditional 3D-bioprinting methods do not allow for. Studies have shown that hydrogels can be designed with different compartments that are loaded with signaling molecules, growth factors, or functionalized fibers. The present invention has found that neuronal organoids can be loaded in these compartments, resulting in regional patterning of cortical tissue based on the applied gradients of the signaling factors. Hydrogel and/or Pluronic can be bioprinted to create the compartments and neuronal spheroids can be patterned using the method of the present invention.

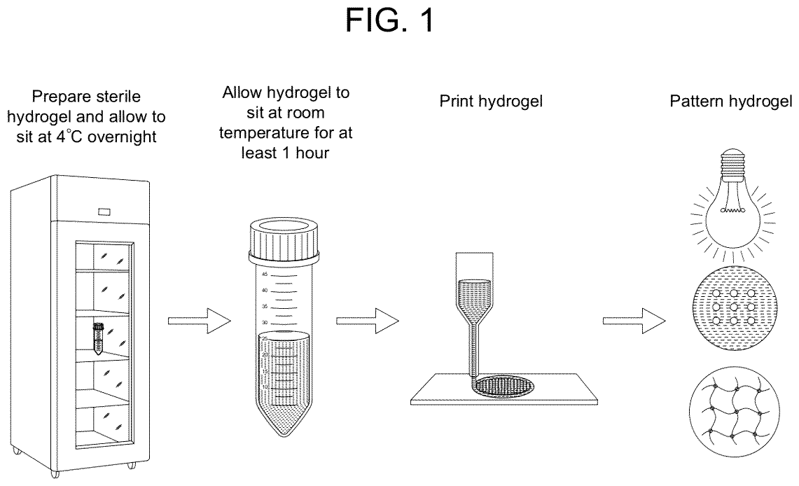

The basic process of preparing hydrogels for printing according to the present invention is shown in FIG. 1. The printed hydrogel is micropatterned using a photolithographic approach. Then, in one embodiment, human neurons are seeded on the hydrogels and cultured. The resulting structure is a neuronal model which is useful for many lines of research, including understanding the blood brain barrier. Aspects of the present invention are described below.

### Hydrogels

It has been shown that the rigidity of the microenvironment is a main regulator of morphogenesis in extracellular matrix (ECM) hydrogels and within tissues. Hydrogels composed of Matrigel and collagen, for example, have been used extensively to study the innate capacity of human cells to self-arrange into 3D structures (spheroids and organoids) with defined spatial configuration.

Hydrogels better mimic brain conditions in both the stiffness and the addition of specific extracellular matrix components that can tailored to match physiological conditions. Hydrogels also offer the benefit of offering a 3D environment that cells can infiltrate that traditional 2D culture methods do not allow. Because of the advantages hydrogels offer compared to 2D culture methods, hydrogels provide an ideal modality to utilize.

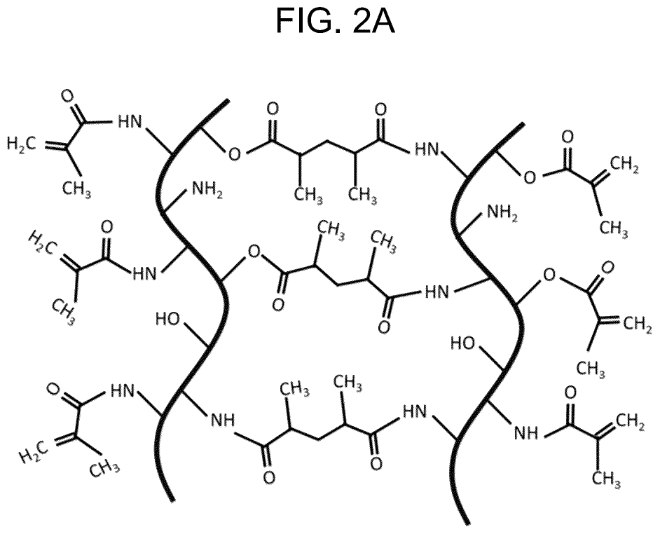

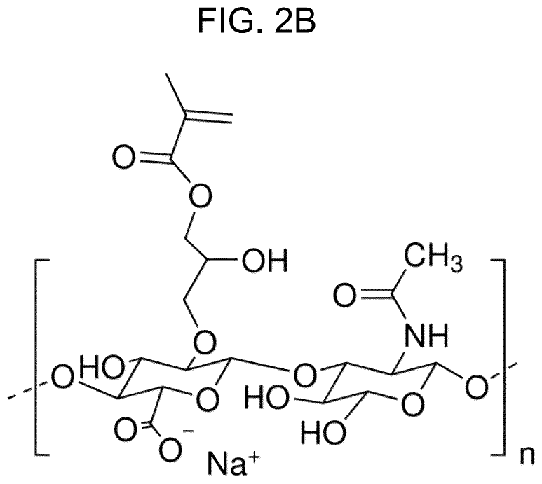

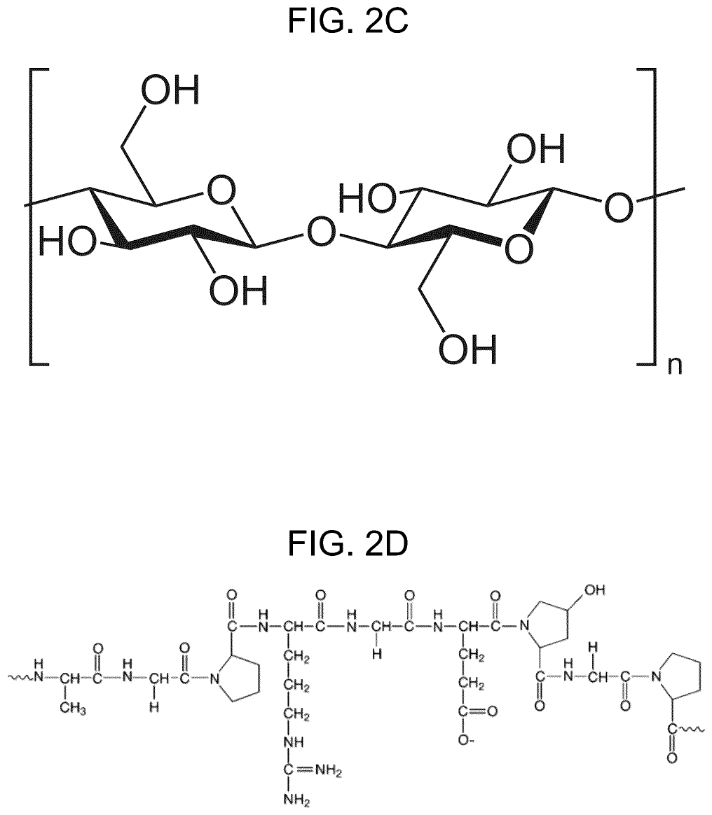

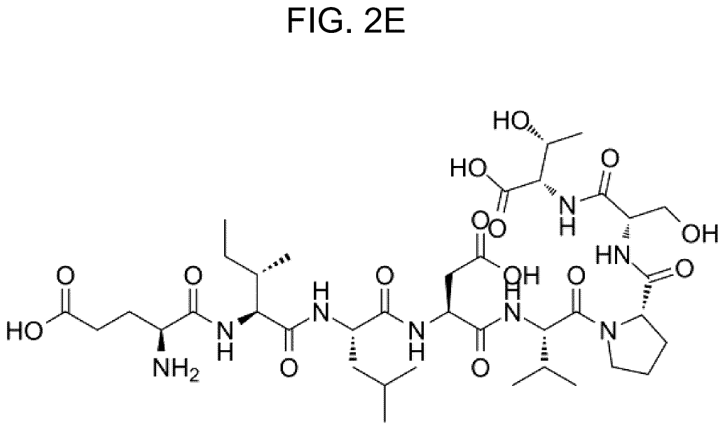

Hydrogel components that are useful in the present invention to recreate the ECM components for microfluidic organ-on-a-chip (OOC) systems include, among other hydrogels, gelatin methacrylate (GelMA), hyaluronic acid methacrylate (HAMA), cellulose, and fibronectin (FN). The chemical structures of these compounds are shown in FIGS. 2A-2E.

Gelatin and GelMA are two of the most used materials as main components in hydrogels used in bioprinting. Gelatin is a water-soluble protein derived from the partial hydrolysis of collagen. Gelatin is often combined with other hydrogel components such as alginate, chitosan, and fibrinogen, with gelatin-based hydrogels displaying excellent biocompatibilities and rapid biodegradations. Bovine-derived gelatin has been used in regenerative medicine for several decades. The presence of the arginine-glycine-aspartic acid (RGD) peptide sequence which promotes cell adhesion. Changing the accompanying components of gelatin-based hydrogels can significantly alter the final properties of 3D printed hydrogels. The mechanical properties of physically crosslinked gelatin-based hydrogels mainly depend on the main gelatin solution. Transglutaminase (Tg) may also be added to crosslink the gelatin and make it more temperature stable which is ideal for printing.

To improve the mechanical properties and stability of gelatin hydrogels, the gelatin may be blended with methacrylate to create GelMA. This blend improves the viscosity of the resulting hydrogel and photocrosslinking of GelMA with a photoinitiator such as LAP and ultraviolet (UV) light can significantly increase the structural stability of the printed 3D structure. The RGD sequence and biocompatibility properties are not influenced by the addition of the methacrylate group, so GelMA hydrogels also display good cell compatibility.

Hyaluronic acid (HA) is a major structural role in the brain's ECM. HA is responsible for wound healing, tissue formation regulation, inflammation, and morphogenesis due to its high affinity to adhesion receptors. However, HA-based hydrogels display poor cell adhesion, limiting their application to cell-based hydrogel experiments. HA is another compound that methacrylate is added to form HAMA. HAMA does offer resistance to enzymatic degradation compared to unmodified HA and remains biocompatible. Hydrogels that combine GelMA and HAMA are useful in the present invention, since they possess excellent biocompatibility and chemical-physio properties. The addition of HAMA to GelMA hydrogels improves the stability of the hydrogel.

The hydrogels of the present invention have the following benefits when compared to other existing materials: they are biocompatible, transparent, and suitable for traditional microscopic techniques. The hydrogels of the present invention can be used for bioprinting of living single and aggregate cells (spheroids, organoids). They can be combined with fibrin or other organic or synthetic biomolecules to better recapitulate tissue biology of specific organs. They can also be combined with alginate or methacrylate molecules to allow for chemical or UV-mediated crosslinking after bioprinting and to achieve high-resolution 3D-patterns. The patterns can be functional as demonstrated by our ability of perfusing medium through them and to seed other cells within the micro-channels generated via chemical or UV crosslinking.

### Bioprinting the Hydrogel

Bioprinting is utilized in the present invention to create a thin hydrogel layer for repeatable patterning before cell seeding. Bioprinting refers to the tissue engineering technique of 3D printing cells, growth factors, and other biomaterials, often with the goal to mimic natural tissue. With bioprinting, the biological material is deposited layer by layer to create a defined structure. Extrusion-based bioprinting is often used due to its low cost and versatility to work with a variety of materials including hydrogels, biocompatible copolymers, and cell spheroids that have a wide range of viscosities and densities. With extrusion-based printing of hydrogels, cells and spheroids may be incorporated in the bioink and extruded in the shape of the printed structure.

### Micropatterning of the Hydrogel

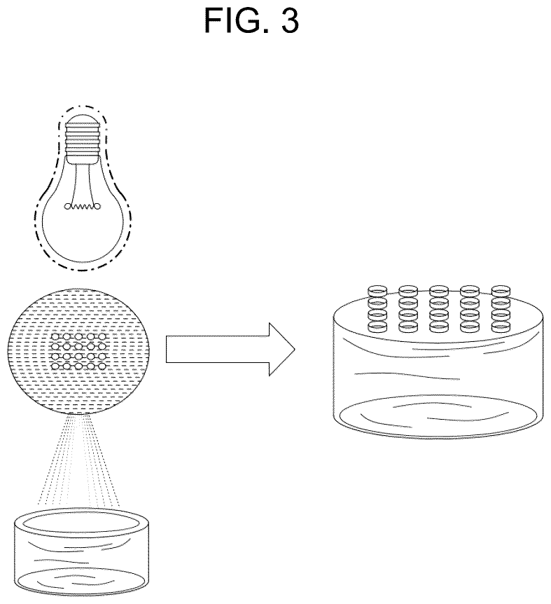

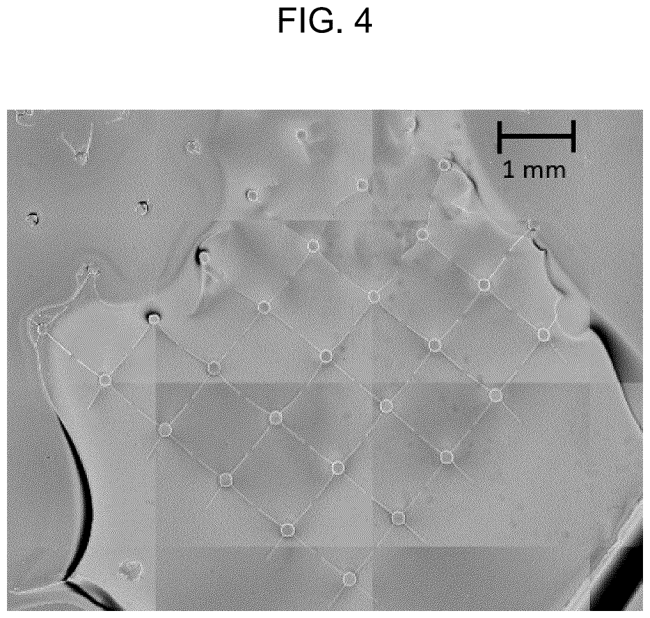

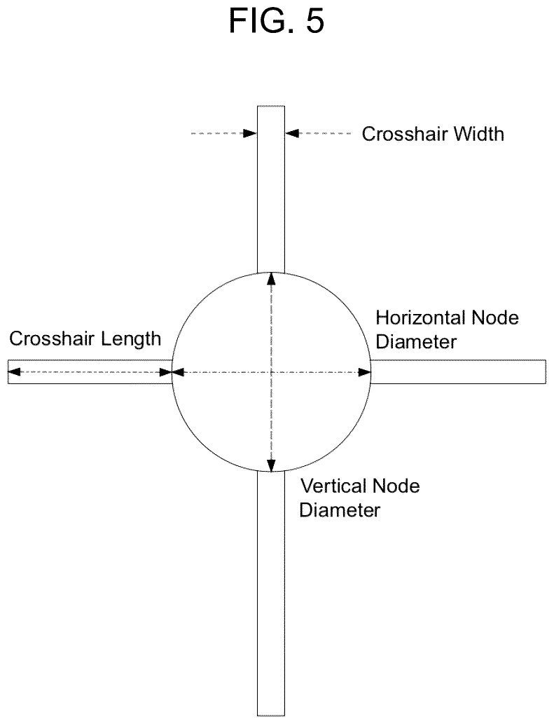

One aspect of the present invention is the use of a photolithographic approach to patterning hydrogels that guides synaptic formation. Referring to FIG. 3, a photomask is located over a hydrogel. In an embodiment of the present invention, the surface of a hydrogel is patterned using a photomask with an ultraviolet (UV)-crosslinkable hydrogel under UV light. In another embodiment, the surface of a hydrogel is patterned using a photomask and a sacrificial material. In one embodiment, a sacrificial bioink such as pluronic is used to create the micropattern. The photomask is then removed, resulting in a micropatterned hydrogel. FIG. 4 is an example of such a hydrogel. It is an image of a micropatterned HAMA-GelMA-Matrigel hydrogel. Micropatterned hydrogels can be imaged to evaluate dimension measurements. For example, an Olympus IX73 microscope can be used to image the pattern. The images can be used to measure the pattern dimensions to determine how accurately the crosslinked hydrogels are to the photomask's dimensions. The dimensions measured are the horizontal and vertical diameters of the nodes, the crosshair lengths, and the distal crosshair widths (see FIG. 5).

The independent variables that may be modified for the present invention include the micropattern dimensions, the hydrogel parameters, and the crosslinking parameters. Patterns that have been shown to guide synaptic formation tend to require a resolution of 10 μm which is one of the main reasons why a photolithographic approach was chosen as opposed to a bioprinting approach. In one embodiment, neurons are cultured on a 2.5% gelatin methacrylate (GelMA, Sigma-Aldrich)/4 mg/ml Matrigel hydrogel. Induced pluripotent stem cell-neurogenin 2 (iPSC-NG2) derived neurons will adhere to this hydrogel and remain viable for at least three days. A protocol was established to create the patterns with the hydrogel and then determine the contribution of micropatterned surfaces to synaptic network formation.

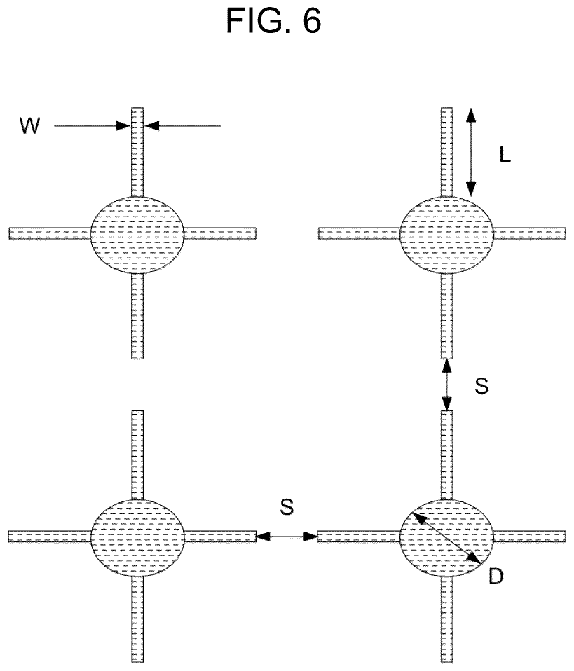

In one embodiment of the present invention, photomasks were created with patterns that have been shown to guide synapse formation in literature. The three photomasks have the same basic node-crosshair pattern with different node diameters and crosshair lengths (see FIG. 6 and Table 1) with 100 μm between crosshairs. This established a method that allows the patterns to be consistently reproduced before producing the hydrogels that neurons were cultured on.

TABLE 1
Pattern dimensions

| Node Diameter | Crosshair Length | Crosshair Width | Crosshair Spacing |
|---|---|---|---|
| (μm) (D) | (μm) (L) | (μm) (W) | (μm) (S) |
| 250 | 450 | 10 | 100 |
| 150 | 450 | 10 | 100 |
| 50 | 300 | 10 | 100 |

### Neuronal and Astrocyte Seeding

Research groups have demonstrated the ability to create vascularized OOC models that reflect some aspects of the BBB, including neuronal activity. However, none of the previous models allow monitoring of neuronal activity at the level of single cells or synapses due to the difficulty in identifying pre-synaptic and post-synaptic regions. Neurons in culture inherently display little organization, making synapses hard to identify. These previous models are unable to capture the directionality of neuronal pathways which is a key feature of the nervous system organization and may provide targets for new treatments.

Many neuronal microfluidic models also utilize polydimethylsiloxane (PDMS) which is hydrophobic. Many neurotransmitters are also hydrophobic such as glutamate and can be absorbed by the construct, which may affect measurable levels of neurotransmitters in the model, which in turn would not accurately reflect the physiological conditions.

Understanding the intercellular cross talk that occurs in the BBB can help in better understanding normal tissue and the tissue's response to ischemic strokes. Identifying differences in normal vs post-stroke neuronal activity can lead to the development of better treatments and prevention plans that reduce the negative side effects of tPA and avoid the need for invasive surgical procedures. The ideal stroke treatment would prevent the long-term disruption of the BBB that tPA can cause and would not be neurotoxic which would reduce the long-term medical costs for stroke patients. To be able to study neuronal signaling to identify differences in neurodegenerative-diseased tissues such as post-stroke tissue, it is important to guide the formation of functional synaptic structures to create clear synapses and provide a physiologically relevant environment for the cells.

In one embodiment of the present invention, Matrigel is applied to the hydrogel to improve cell adhesion. For example, Gelatin methacrylate (GelMA) can be combined with Matrigel to support growth and survival of hPSC-derived neuronal progenitors that remain viable for over 2 weeks. GelMA is one of the most widespread biomaterials used in tissue-engineering. Several human cell types including epithelia and endothelial cells have been previously shown to adhere and grow on 3D scaffolds made of GelMA. However, neurons cannot spread on the surface of a scaffold made only with GelMA and they tend to form clumps. Cell-adhesion factors included in the Matrigel allows neurons to adhere and spread on the scaffold photo-crosslinked via UV-light. Neurons do not adhere to the surface of UV-crosslinked scaffolds obtained via a combination of GelMA and Alginate, a sugar polymer frequently used in tissue-engineering. The present invention has found that the combination of GelMA and Matrigel not only allows for neuronal cell adhesion, but it also supports encapsulation and sustains neuronal cell growth inside the scaffold for over a week.

### Spheroids

Hydrogels of the present invention can be used to bioprint living spheroids. Specifically, hydrogels that contain GelMA/HAMA ad Gelatin-TG or Cellulose/GelMA and Gelatin-TG can both be used for bioprinting of living cancer spheroids and organoids. Interestingly, spheroids growing in the hydrogels of the present invention maintain a hypoxic core, which is generally found within the mass of solid tumors such as glioblastoma and others. This is critical to sustain cancer cell stemness, drug resistance and regeneration.

### Materials and Methods

### Photomask Manufacturing

Photomasks with patterns that have been shown to guide synapse formation in literature were created using Computer Aided Design (CAD) software. The photomasks were then manufactured by a third party (Output City CAD/Art Services). The three photomasks have the same basic node-crosshair pattern with different node diameters and crosshair lengths (see FIG. 3) with 100 μm between crosshairs and occur in a 15×15 array.

### Gel Preparation and Photopatterning Evaluation

The preparation of gelatin-alginate hydrogel (“Gelatin-TG”) is a 2-day process. On the first day the sodium alginate (10%) is mixed and allowed to mix overnight to remove any unmixed lumps present. The next day gelatin (2.5%) and transglutaminase (TG) (5%) is added and mixed thoroughly. Once the mixture is ready the hydrogel, the pH of the hydrogel is checked and brought to 7 after which it is autoclaved to sterile for cells to be seeded in. The autoclaved hydrogel is aliquoted to prevent contamination of the whole batch and preserve the properties of the hydrogel for longer.

Hydrogel and crosslinking parameters were tested to identify a method to reproduce three photomask patterns. The main parameters for testing were the time of UV exposure, the distance between the photomask and the hydrogel (direct vs indirect contact), and the amount and thickness of the hydrogel.

The other parameters that are important and may be recorded include the distance between the photomask and the UV light, the removal method of the photomask from the hydrogel in the case of direct contact, and the temperature of the hydrogel before and after UV exposure. Initial photopatterning attempts were performed on glass slides and the success of creating the pattern was quantitative (pattern present or absent). The parameters that were tested and the success of creating the pattern for each run were recorded.

Initial patterning attempts were performed on GelMA and/or HAMA hydrogels. Gradually, other hydrogel components were added and patterned to the GelMA and HAMA. Successful patterns were imaged to evaluate dimension measurements. An Olympus IX73 microscope was used to image the pattern. The images were used to measure the pattern dimensions to determine how accurately the crosslinked hydrogels are to the photomask's dimensions. The dimensions measured are the horizontal and vertical diameters of the nodes, the crosshair lengths, and the distal crosshair widths (see FIG. 5). The dimensions of n=24 node-crosshair constructs were measured for each hydrogel.

### EXAMPLES

### Example 1-Hydrogel Formulations

The following hydrogel concentrations and components were prepared: Gelatin-HAMA, Gelatin-Cellulose, HAMA-Gelatin-Cellulose, HAMA-Gelatin-Tg0.1%-Cellulose, GelMA-Gelatin-Tg0.1%-Cellulose, GelMA-Gelatin-Tg0.1%-HAMA, and GelMA-Gelatin-Tg0.3%-Cellulose.

During preparation, the gelatin hydrogel is neutralized, and the pH is measured and adjusted to reach neutrality (about 7) before the Tg is added. The hydrogels were prepared for printing using a 2-day process as shown in FIG. 1. On the first day, the hydrogel components are mixed and allowed to sit overnight at 4° C. to allow the gel to become stable. The next day, the hydrogel is allowed to sit at room temperature for an hour to allow it to become slightly more viscous for printing of hydrogels.

The hydrogels were tested for printing fidelity to determine if a thin, controlled layer could be printed. Hydrogels that were printable were printed in a grid pattern to determine the fidelity of the hydrogels. The diagonal of the grid consisted of squares with increasing dimensions that were used to determine the fidelity of the print after optimization of printing parameters (1×1 mm, 2×2 mm, 3×3 mm, 4×4 mm, 5×5 mm). Pluronic was also printed using the grid as a control group. N=3 grids per hydrogel type were printed and imaged. ImageJ was then used to measure the inner perimeter of the grids' squares and a Pr value was calculated according to Formula 1:

$$Pr = \frac{Measured Inner Perimeter}{Actual Inner Perimeter from CAD} \qquad Formula 1$$

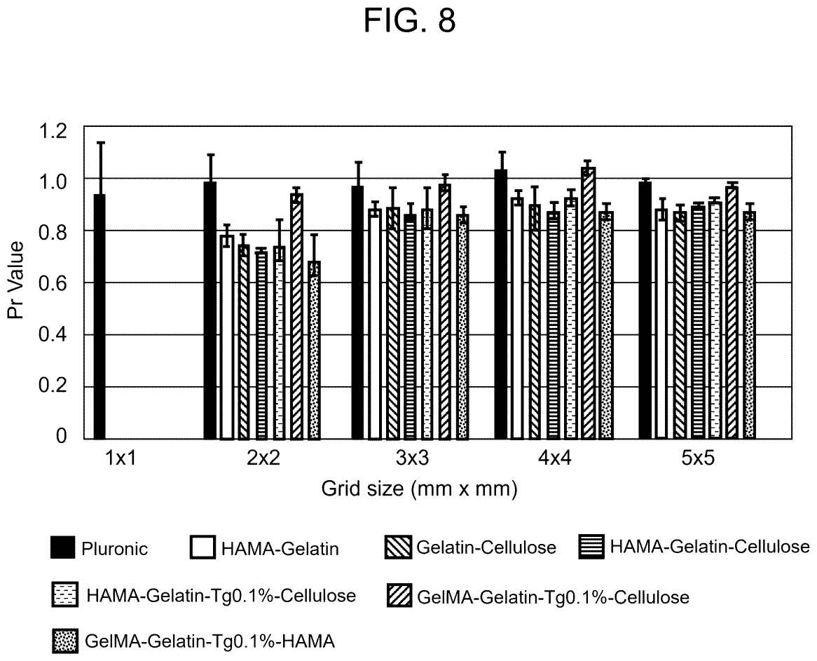

FIG. 8 shows a statistical analysis of printing fidelity showing mean+/−SD. Seven of the eight bioinks/hydrogels that were tested were printable, but the pluronic was the only bioink that was able to print a 1×1 mm square within the grid. It was determined that based on the printability and patternability of the tested hydrogels, GelMA-Gelatin-Tg0.1%-cellulose and GelMA-Gelatin-Tg0.1%-HAMA were particularly useful for their stability and viability.

### Example 2-Bioprinting the Hydrogel

Various hydrogels were printed to assess their printability. Factors to be considered regarding printing conditions for a given hydrogel are the hydrogel composition, the nozzle diameter, the printing pressure, and the speed of the printer head. For each hydrogel type, the printing parameters were first optimized and then n=3 grids/hydrogel were printed and imaged. ImageJ was used to measure the inner perimeter of the squares within the grids and a Pr value (measured value divided by nominal perimeter) was used to determine the fidelity of the printed hydrogel. Pluronic, which is typically used as a sacrificial bioink in bioprinting, was used as a control comparison group. Table 2 shows the conditions and preferred printing parameters used to achieve a printable resolution:

TABLE 2
Printing Parameters

| Hydrogel | Pressure | | Speed | Nozzle |
|---|---|---|---|---|
| Gelatin-HAMA | 60 | kPa | 2 m/s | 25G blunt |
| Gelatin-Cellulose | 50 | kPa | 2 m/s | 25G blunt |
| HAMA-Gelatin- | 60 | kPa | 2 m/s | 25G blunt |
| Cellulose | | | | |
| HAMA-Gelatin- | 85 | kPa | 2 m/s | 25G blunt |
| Tg0.1%-Cellulose | | | | |
| GelMA-Gelatin- | 185 | kPa | 2 m/s | 25G blunt |
| Tg0.1%-Cellulose | | | | |
| GelMA-Gelatin- | 75 | kPa | 2 m/s | 25 G conical |
| Tg0.1%-HAMA | | | | |
| GelMA-Gelatin- | Would not print (pressure up to max 200 kPa) | | | |
| Tg0.3%-Cellulose | | | | |
| Pluronic | 180 | kPa | 2 m/s | 25G blunt |

### Example 3-Hydrogel Patterns

The present invention has identified parameters that allow patterns shown to guide axon alignment to be created with hydrogels. Preliminary cell viability studies indicate that the long UV crosslinking time should not negatively affect cell survival and the GelMA-Matrigel and GelMA-Matrigel-HAMA hydrogels seem to be best for cell adhesion and spreading.

TABLE 3
Parameters for Matrigel-Based Hydrogels

| Parameters | Optimized Parameter |
|---|---|
| Mask Distance from Hydrogel | Direct contact |
| UV Exposure Time | 90 s (vs 20 s for non-Matrigel hydrogels) |
| Temperature of Hydrogel | Room Temp |
| Hydrogel Volume | 150-200 μl |

TABLE 4
Pattern Can Be Recapitulated with
HAMA-GelMA-Matrigel Hydrogel

| | Nominal | | Measured |
|---|---|---|---|
| Pattern | Dimension | n | Dimension: Mean ± |
| Feature | (μm) | Measured | SD (μm) |
| Vertical Diameter | 250 | 16 | 0.2009 ± 0.0119 |
| Horizontal | 250 | 16 | 0.2053 ± 0.0096 |
| Diameter | | | |
| Crosshair Length | 450 | 64 | 0.4589 ± 0.0184 |
| Crosshair Width | 10 | 64 | 0.0163 ± 0.0037 |

### Example 4-Biocompatibility

200 μl of each hydrogel were pipetted into n=3 wells. Two hydrogels with fibronectin were pipetted into six total wells-one set of n=3 wells were crosslinked for 30 s and the second set of n=3 wells were crosslinked for 90 s to determine if the longer crosslinking time has any effect on cell adhesion and proliferation. The other hydrogels were crosslinked for 30 s.

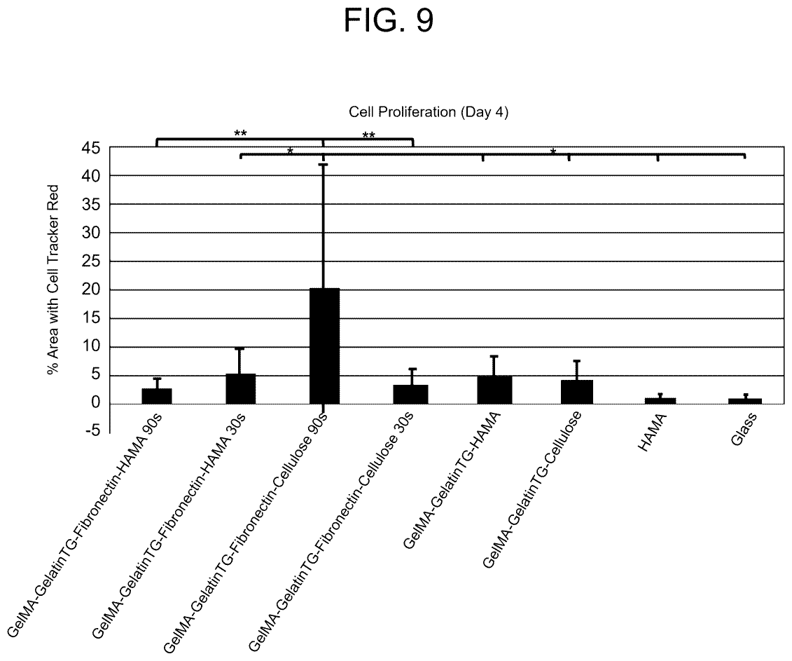

For the initial biocompatibility testing, human U87 MG cells were purchased from ATCC. 200 μl of hydrogel was crosslinked in each well of a 6 well chamber slide. U87 MG cells were suspended a density of 200,000 cells per ml and 200 μl of cell suspension was added to each well. The cells were stained with DAPI and cell tracker red and imaged. After incubating in the incubator at 37° C. and 5% CO2 for four days, the hydrogels were again imaged. 3-12 images were taken per hydrogel condition (n=1-4 images per well) and the percent area of cell tracker red fluorescence was measured with ImageJ to determine adhesion and viability of the cells. FIG. 9 shows cell tracker red staining of U87 MG cells on hydrogels looking at viability (day 4). The results indicate that the GelMA-gelatin-Tg0.1%-fibronectin-cellulose hydrogel had significantly higher percent areas with cell tracker red than any of the other conditions. There was a very large standard deviation (SD) though, so the average 20% area does not accurately reflect high cell tracker red for all photos. There were no other significant differences between any of the other groups. The 1% HAMA hydrogel had very low adhesion and proliferation of cells after 4 days as HA does not contain poor cell adhesion.

### Example 5-Neuronal and Astrocyte Seeding

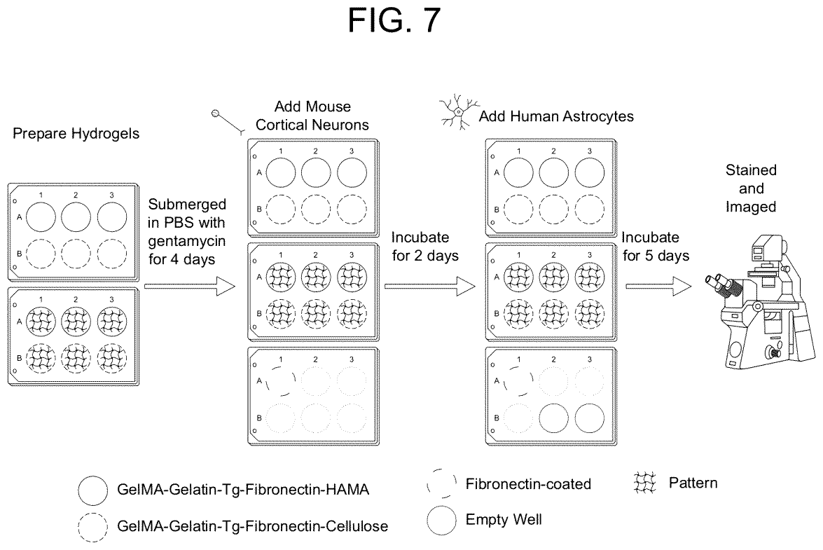

FIG. 7 summarizes a method according to the present invention for the neuronal and astrocyte seeding. In one embodiment, for the neuronal seeding GelMA-Gelatin-Tg-Fibronectin-Cellulose and GelMA-Gelatin-Tg-Fibronectin-HAMA hydrogels were prepared and transferred to sterile 3 ml syringes due to technical issues with the bioprinter and the hydrogel was extruded into a thin square with a 25G nozzle in a 6 well plate. The same patterning technique described in the previous section was used to pattern the hydrogels. n=3 patterned hydrogels were prepared per hydrogel type.

n=3 non-patterned hydrogels were also prepared. A mask that did not contain any pattern (100% transparent) was placed over the extruded hydrogels and the same protocol above was followed to prepare the non-patterned hydrogels. An additional well coated with fibronectin (0.2 mg/ml) was also prepared.

4 ml of PBS with gentamycin was added to each well to submerge the crosslinked hydrogels and the hydrogels were left in the incubator at 37° C. and 5% CO2 for five days.

For neuronal seeding, rat cortex neurons purchased from Gibco were seeded on micropatterned hydrogels at a density of 300,000 cells/ml and placed in the incubator at 37° C. and 5% CO2.

On day 2, there were very few neurons remaining on the hydrogels. Therefore, human astrocytes purchased from Cell Science were also seeded onto the hydrogels and fibronectin-coated well. Astrocytes were also seeded in n=2 empty wells. After five days post-seeding of the astrocytes, the cells were stained with Calcein and Hoechst. The fibronectin and empty wells were control groups to check if there might be any issues with the cells due to moving labs and transporting the cells.

### Example 6-Synapse Formation

To obtain functional insights into the process of synapses formation in vitro, human neurons were seeded on micropatterned hydrogels (minimum n=3 per pattern) and non-micropatterned hydrogels (control group) and these cells were cultured for 21 days (when synaptic networks should form). At 21 days post-seeding, live calcium imaging was performed to verify the functionality and synchronization of the synapses. Cell viability and synaptic region staining was also carried out to quantify neuron viability and the number of distinct synapses that were formed. This was done to compare the organization of the synaptic networks between the patterns and to the non-patterned hydrogel and to evaluate if micropatterning allows the directionality of neuronal signaling to be observed. It was found that, when compared to cells growing on a flat (not patterned) surface, neurons growing on a micropatterned surface display higher calcium signaling (firing) activity.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A device for modelling physiological and pathophysiological states of a brain comprising a hydrogel wherein the hydrogel comprises micropattern channels having a length, width and depth and further, wherein the micropattern channels comprise cells selected from the group consisting of neurons, astrocytes, microglia, oligodendrocytes, neuronal organoids, cancer cells, cancer spheroids, brain tumoral cells and combinations thereof.

2. The device of claim 1 wherein the cells are either of primary origin or derived from stem cells.

3. The device of claim 1 wherein the micropattern channels comprise human neurons and the human neurons form a synaptic network.

4. The device of claim 3 wherein the human neurons form a synaptic network that is co-cultured with tumoral cells, wherein the tumoral cells are human or mice tumoral cells.

5. The device of claim 1 wherein the micropattern channels further comprise endothelial cells.

6. The device of claim 1 wherein one or more of the micropattern channels have inlets that allow for direct access to the channels with conventional micropipettes.

7. The device of claim 1 wherein the hydrogel comprises parallel microfluidic chambers.

8. The device of claim 7 wherein the hydrogel comprises multiple parallel microfluidic chambers.

9. The device of claim 1 wherein the hydrogel is selected from the group consisting of Gelatin-HAMA, Gelatin-Cellulose, HAMA-Gelatin-Cellulose, HAMA-Cellulose-Gelatin-TG, GelMA-Cellulose-Gelatin-TG, and GelMA-HAMA-Gelatin-TG.

10. The device of claim 1 wherein the hydrogel is GelMA-HAMA-Gelatin-TG.

11. The device of claim 1 wherein the hydrogel material further comprises a material that improves cell adhesion.

12. The device of claim 1 wherein the hydrogel material further comprises Matrigel, collagen, fibronectin or combinations thereof.

13. The device of claim 1 wherein the micropattern channels have a width of less than about 20 μm.

14. The device of claim 1 wherein the micropattern channels have a width of less than about 10 μm.

15. A device for modelling physiological and pathophysiological states of a brain comprising a hydrogel wherein the hydrogel comprises micropattern channels having a length, width and depth and further, wherein the micropattern channels comprise endothelial cells, and further, wherein the hydrogel has an apical surface and the apical surface comprises cells selected from the group consisting of neurons, astrocytes, microglia, oligodendrocytes, neuronal organoids, cancer cells, cancer spheroids, brain tumoral cells and combinations thereof.

16. The device of claim 15 wherein the endothelial cells form a tight monolayer and physiologically relevant barrier function and can be perfused with cell culture medium or blood.

17. A method of making a micropatterned hydrogel comprising:
a. printing a hydrogel material on a substrate;
b. positioning a photomask over the hydrogel material;
c. exposing the photomask and hydrogel to UV light; and
d. removing the photomask;
wherein micropattern channels are created on the surface of the hydrogel material, said micropattern channels having a length, width and depth.

18. The method of claim 17 wherein the hydrogel is selected from the group consisting of Gelatin-HAMA, Gelatin-Cellulose, HAMA-Gelatin-Cellulose, HAMA-Cellulose-Gelatin-TG, GelMA-Cellulose-Gelatin-TG, and GelMA-HAMA-Gelatin-TG.

19. The method of claim 17 wherein the hydrogel material further comprises a material that improves cell adhesion.

20. The method of claim 19 wherein the hydrogel material further comprises Matrigel collagen, fibronectin or combinations thereof.

21. The method of claim 17 wherein the hydrogel material is bioprinted.

22. A method of making a neuronal activity model comprising:
a. making a micropatterned hydrogel according to claim 17; and
b. seeding cells in one or more of the micropattern channels;
wherein the cells are selected from the group consisting of neurons, astrocytes, microglia, oligodendrocytes, neuronal organoids, cancer cells, cancer spheroids, brain tumoral cells and combinations thereof.

23. The method of claim 22 wherein the cells are human neurons, and further, wherein the human neurons form a synaptic network.

24. A method of making a micropatterned hydrogel comprising:
a. printing a hydrogel material on a substrate;
b. applying a sacrificial bioink to the hydrogel material;
wherein the sacrificial bioink creates micropattern channels on the surface of the hydrogel material, said micropattern channels having a length, width and depth.

25. The method of claim 24 wherein the sacrificial bioink is pluronic.

26. The method of claim 24 wherein the hydrogel is GelMA-HAMA-Gelatin-TG.
